(19) United States
(12) Patent Application Publication
(10) Pub. No.: US 2020/0365243 A1
(43) Pub. Date: Nov. 19, 2020

(54) SYSTEM AND METHOD FOR AUTOMATED RETRIEVAL AND ANALYSIS OF MEDICAL RECORDS

(71) Applicant: KONINKLIJKE PHILIPS N.V., EINDHOVEN (NL)

(72) Inventors: CHRISTINE MENKING SWISHER, SAN DIEGO, CA (US); SHEIKH SADID AL HASAN, CAMBRIDGE, MA (US); MINNAN XU, CAMBRIDGE, MA (US); RAYMOND CHAN, SAN DIEGO, CA (US); OLADIMEJI FEYISETAN FARRI, YORKTOWN HEIGHTS, NY (US); MAYA ELLA BARLEY, WATFORD (GB); ANDREW G HOSS, CAMBRIDGE, MA (US)

(21) Appl. No.: 16/639,779

(22) PCT Filed: Aug. 30, 2018
(86) PCT No.: PCT/EP2018/073287

§ 371 Date: Feb. 18, 2020

Related U.S. Application Data
(60) Provisional application No. 62/558,035, filed on Sep. 13, 2017.

(51) Int. Cl. G16H 10/60; G16H 80/00

(52) U.S. Cl. CPC G16H 10/60; G16H 80/00

(57) ABSTRACT

A record collection device (201, 400) configured to collect records for an individual (452). The device includes: a user interface (440) configured to receive input from the user, a location module (410) configured to detect a location the device; and a processor (420) configured to: (i) automatically determine, by a processor of the record collection device, that the detected location corresponds to a location where a record may be generated or stored; (ii) receive, from the individual via the user interface, approval to request the record from the determined location; and (iii) send, in response to the individual's input, a request to the determined location for the individual's record.

### FIELD OF THE INVENTION

The present disclosure is directed generally to methods and systems for automatically obtaining medical records.

### BACKGROUND

Health information such as medical records typically resides in many different locations rather than in a single, easily-accessible centralized location. This fragmentation of health information can have serious negative consequences. For example, fragmentation of health information across different care settings and between specialties limits the use of prior medical history in clinical decisions. Additionally, health information from home or primary care settings are not readily available in hospital and acute settings, such as during an emergency situation. Additionally, because patients do not have access to their health information, they are not empowered to manage their own health care.

Accordingly, the medical industry lags behind other industries in the delivery of data informatics. When a patient is visiting a specialist, it is not uncommon for the patient to hand-deliver medical records or recite their medical history from memory. Relying on the patient or caregiver's memory results in records being inaccurate or incomplete, especially for patients with complex diseases or comorbidities.

Extraction and normalization of health information from medical records is complex and time-consuming. Format and source type vary widely, and many private practices have yet to adopt electronic records. This challenge limits access to health data for patients, clinicians, and researchers. Current methods to interpret and normalize various data sources require specialized adapters for each data source format, which is not scalable. Lack of a scalable data interpretation mechanism makes it difficult to utilize them across care settings and has made precision health and integrated data tools difficult to deploy and develop.

### SUMMARY OF THE INVENTION

There is a continued need for methods and systems that automatically obtain and analyze medical records to improve access to health information.

The present disclosure is directed to inventive methods and systems for obtaining, sharing, and/or analyzing medical records. Various embodiments and implementations herein are directed to a system that obtains an individual's medical records using a location-aware medical record collection device such as a smartphone or a wearable device. The record collection device automatically detects the location of the individual and determines that the location corresponds to a location where a medical record may be generated or stored. Upon approval from the user, the record collection device sends a request to the determined location for the individual's medical record. The record collection device may also record conversations with the health care professional. The record collection device may also send one or more of the individual's medical records to the location, thereby enabling sharing of important medical information.

Generally in one aspect, a record collection device configured to collect records for an individual is provided. The device includes: a user interface configured to receive input from the user, a location module configured to detect a location of the device; and a processor configured to: (i) automatically determine, by a processor of the record collection device, that the detected location corresponds to a location where a record may be generated or stored; (ii) receive, from the individual via the user interface, approval to request the record from the determined location; and (iii) send, in response to the individual's input, a request to the determined location for the individual's record.

According to an embodiment, the device further includes a form generator configured to generate a release form or authorization form for the records, wherein the sent request comprises the generated form.

According to an embodiment, the device further includes a microphone, and the processor is further configured to record a conversation between the individual and a healthcare provider at the location. According to an embodiment, the processor is configured to send the recorded conversation to a remote server or database.

According to an embodiment, the processor is configured to send, in response to input from the individual, one or more of the individual's records to the determined location.

According to an embodiment, the processor is configured to send an authorization request to the individual seeking approval to request the record from the determined location.

According to an embodiment, the device is in communication with a database comprising information about a plurality of locations where records are generated or stored.

According to an aspect, a method for obtaining an individual's record using a record collection device is provided. The method includes the steps of: (i) automatically detecting, via the record collection device, a location of the individual; (ii) automatically determining, by a processor of the record collection device, that the location corresponds to a location where a record for the individual may be generated or stored; (iii) receiving, from the individual, approval to request the generated or stored record from the determined location; and (iv) sending, in response to the individual's approval, a request to the determined location for the individual's record.

According to an embodiment, the method further includes the step of receiving, by the record collection device, the requested record. According to an embodiment, the method further includes the step of sending the received record to a remote server or database.

According to an embodiment, the method further includes the step of generating a release form or authorization form for the record, where the sent request comprises the generated form.

According to an embodiment, the method further includes the step of recording, using a microphone of the record collection device, a conversation between the individual and a healthcare provider at the location.

According to an embodiment, the method further includes the step of sending, in response to input from the individual, one or more of the individual's records to the determined location.

According to an embodiment, the method further includes the step of extracting information from a received record. According to an embodiment, the method further includes the step of generating a timeline from the received record.

According to an embodiment, the method further includes the step of sending an authorization request to the individual, wherein the authorization request seeks approval to request the record from the determined location.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects of the present invention discussed herein. The terms “program” or “computer program” are used herein in a generic sense to refer to any type of computer code (e.g., software or microcode) that can be employed to program one or more processors or controllers.

The term “network” as used herein refers to any interconnection of two or more devices (including controllers or processors) that facilitates the transport of information (e.g. for device control, data storage, data exchange, etc.) between any two or more devices and/or among multiple devices coupled to the network. As should be readily appreciated, various implementations of networks suitable for interconnecting multiple devices may include any of a variety of network topologies and employ any of a variety of communication protocols. Additionally, in various networks according to the present disclosure, any one connection between two devices may represent a dedicated connection between the two systems, or alternatively a non-dedicated connection. In addition to carrying information intended for the two devices, such a non-dedicated connection may carry information not necessarily intended for either of the two devices (e.g., an open network connection). Furthermore, it should be readily appreciated that various networks of devices as discussed herein may employ one or more wireless, wire/cable, and/or fiber optic links to facilitate information transport throughout the network.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the invention will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention.

FIG. 1 is a flowchart of a method for obtaining medical records, in accordance with an embodiment.

FIG. 2 is a schematic representation of a display screen of a record collection device, in accordance with an embodiment.

FIG. 3 is a schematic representation of a display screen of a record collection device, in accordance with an embodiment.

FIG. 4 is a schematic representation of a location-aware record collection device, in accordance with an embodiment.

FIG. 5 is a schematic representation of a system for obtaining medical records, in accordance with an embodiment.

### DETAILED DESCRIPTION OF EMBODIMENTS

The present disclosure describes various embodiments of a system for obtaining medical records. More generally, Applicant has recognized and appreciated that it would be beneficial to provide a system that automatically requests and obtains medical records based on a determined location of the user. The system utilizes a medical record collection device such as a smartphone or a wearable device. The record collection device automatically detects the location of the individual and determines that the location corresponds to a location where a medical record may be generated or stored. Upon approval from the user, the record collection device sends a request to the determined location for the individual's medical record. According to an embodiment, the record collection device may also record conversations with the health care professional. According to another embodiment, the record collection device may also send one or more of the individual's medical records to the location, thereby enabling sharing of important medical information.

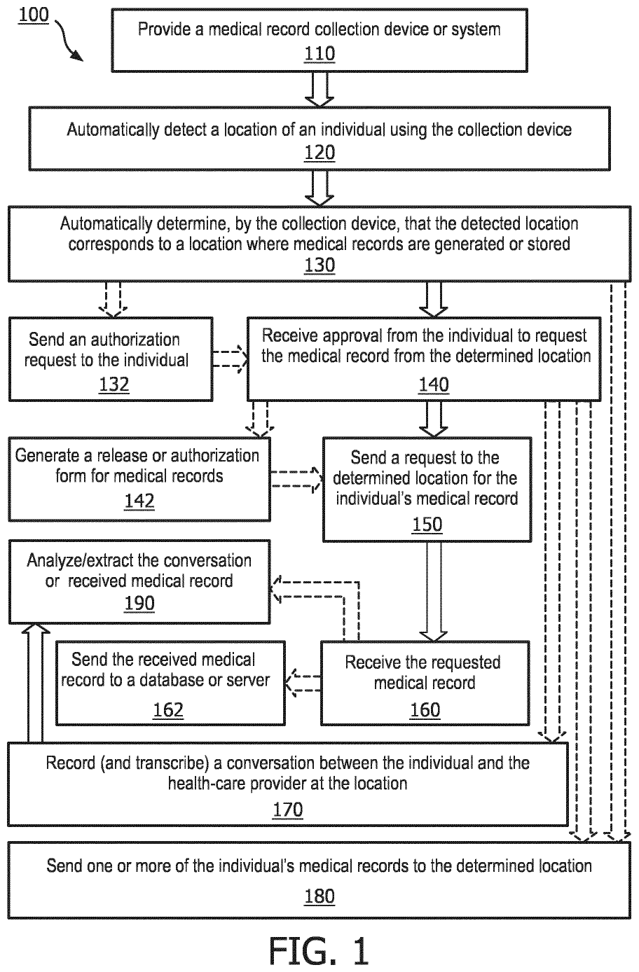

Referring to FIG. 1, in one embodiment, is a flowchart of a method 100 for obtaining medical records. At step 110 of the method a medical record collection device or system is provided. The medical record collection device or system may be any of the devices and/or systems described or otherwise envisioned herein.

At step 120 of the method, the system automatically detects the user's location. According to an embodiment, the system comprises a location-aware or location-determination module, component, or device, such as a global positioning system (GPS) receiver, Bluetooth transceiver, RFID sensor, and/or other module or location-determining method. The location-aware module, component, or device can determine an exact or approximate location and can provide that information to a controller and/or to a user or other remote entity, server, or computer via a communications module. For example, the location-aware module, component, or device can be a GPS receiver that continuously or periodically receives signals utilized to determine a location, and can continuously or periodically provide that information to a user interface and/or to the controller. This allows the system to be location-aware. The system may also utilize, alone or in conjunction with the location-aware or location-determination module, information about the user's schedule to determine location, such as knowledge of a medical appointment in a calendar or other schedule.

At step 130 of the method, the system automatically determines that the detected location corresponds to a location where medical records are generated or stored. The system comprises or is in communication with a database of locations where medical records are generated or stored, such as hospitals, medical offices, laboratories, and many other types of locations. The system may continuously or periodically compare the detected location to the database of locations to identify whenever the user is close to or located at a location where medical records are generated or stored. The system may cross-reference the determined location with the user's schedule, such as knowledge of a medical appointment in a calendar or other schedule, to confirm that the user is at the determined location rather than at a nearby location.

According to an embodiment, steps 120 and 130 of the method are performed automatically by the collection system or device. For example, the collection system or device may, in the background, continuously or periodically determine location and compare that location to the database in order to identify when the user is collocated with a location where medical records are generated or stored. According to an embodiment, the collection device is a smartphone, wearable device, computing device, or other mobile component or device. The collection device may be designed for record collection, or may comprise software such as an application that performs record collection functionality as just one type of functionality performed by the device.

At step 132 of the method, after the collection system or device has determined that the detected location corresponds to a location where medical records are generated or stored, the system or device sends or otherwise presents an authorization request to the user. The authorization request may notify the user that it has tentatively or definitely determined that it is located at a place where medical records are generated or stored, and request input from the user regarding next steps. For example, the authorization request may request that the user authorize a request for medical records, and/or any other request or authorization.

According to an embodiment, this alert may also prompt the user to sign required medical records forms while the user is at the location, and can thus prevent delays and prevent or minimize data security concerns. For example, providing patients with greater control over their data decreases the risk that data will be provided to someone who does not have access. In addition to using established techniques of data encryption and other protection mechanisms, location-aware triggers for health data exchange limit transactions to key moments driven by geographical location and/or the time of medical appointments, thereby adding a level of security. Data may still be shared at other times, but would need to be initiated by either the patient or the care provider or secured by other methods, such as by Blockchain or other methods.

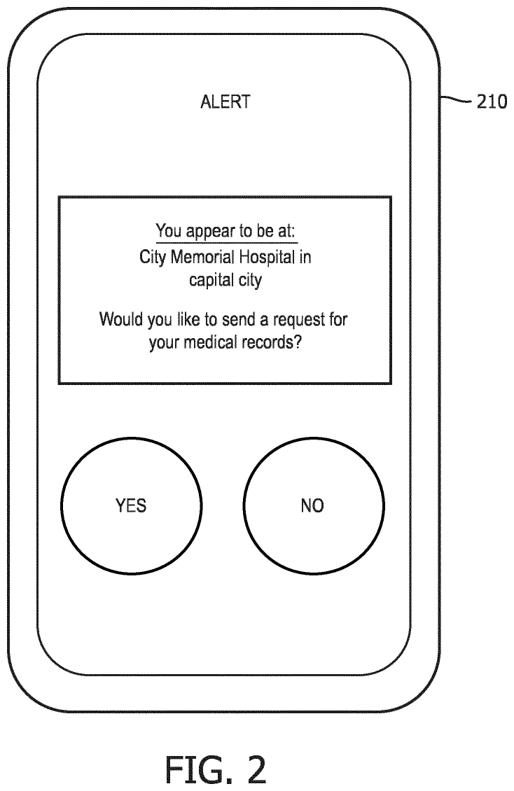

Referring to FIG. 2, in one embodiment, is a smartphone 210 displaying an alert and a request for authorization. The device has tentatively or definitely determined, automatically, that it is located at a hospital where records may be generated or stored, and alerts the user to that fact. The device also optionally presents the user with an authorization request with an option to request records (“YES”) or not request records (“NO”). The user can respond by pressing YES or NO, or by making some other action. According to another example, the device may be a wearable device such as a smartwatch, smart glasses, augmented or virtual reality device, or other device. The user may observe the alert or notification, and may respond by making a motion, voicing a response, or any other method of providing input to the device or system.

At step 140 of the method, the collection system or device receives input from the user approving or authorizing the request to obtain one or more medical records from the detected location where medical records are generated or stored. The input may be a physical motion, an audible response, or any other method of providing input to the device or system. For example, referring to FIG. 2, the response may be pushing YES to authorize the request.

At optional step 142 of the method, the system or device generates a release or authorization form for medical records to accompany the request sent to the location. This can be performed automatically, or in response to input from the user. Automatically generating a release or authorization form, for example, can minimize delays and improve patient/doctor communication. According to an embodiment, the release or authorization need not be a form with labeled text fields, but may be represented by coded transactions using barcodes, RFID or Bluetooth LE transactions (e.g. Apple Pay), QR codes, and other transactions or information.

At step 150 of the method, the record collection system or device sends a request to the determined location for the user's medical records. The request may or may not be accompanied by a release or authorization form. The request may be sent to a predetermined or programmed virtual location or address for such requests. For example, the record collection system or device may comprise or be in communication with a database of contact information for requests for medical records. For example, a location creating or storing medical records may comprise a dedicated contact address, such as an online portal, an email address, fax number, or other contact information, for receiving requests for medical records.

Alternatively, the communication may require an intermediary system or individual, such as a clearinghouse for medical record requests. For example, according to an embodiment, the record collection system or device may not be able to contact the location or may not have contact information for the location. Accordingly, the record collection system or device may utilize a lookup function to find contact information. Additionally or alternatively, the record collection system or device may contact an intermediary such as a third-party individual or service that facilitates this communication. For example, the record collection system or device may be in contact with a free or paid service that facilitates records collection between the record collection system or device and various medical record generation or storage locations.

At step 160 of the method, the record collection system or device receives one or more medical records in response to the request. The record collection system or device may receive the one or more medical records directly from the location or from an intermediary, and may store the received record locally or forward the record to another device or location for storage or analysis. For example, the user may specify, such as in a user setting, that records be received by the device directly. Alternatively, a remote location may receive the one or more medical records, and the record collection system or device may receive only a notification of that receipt. For example, the user may specify, such as in a user setting, that records be received by the device directly, or that records should be sent to an intermediary, clearinghouse, database, or any other location capable of receiving the records.

Accordingly, at optional step 162 of the method, the record collection system or device may forward or otherwise direct the received medical record to a database, remote server, intermediary, clearinghouse, or any other location capable of receiving the received medical record. The record collection system or device may forward the medical record by email, via software installed on the system or device, by reference, or by any other forwarding or communication method. For example, the record collection system or device may not receive the record directly, but may direct a recipient such as an intermediary, clearinghouse, database, or other recipient to send the received record to another location, user, database, or other location.

At optional step 170 of the method, the record collection system or device records and optionally transcribes a conversation between the individual and the health-care provider at the location. This may be an automated feature when the device determines that it is located at a place where medical records are generated or stored, or may be activated in response to user input. For example, the record collection system or device may prompt the user to record audio as the user enters the location, and/or as the device detects a conversation at the location.

According to an embodiment, transcription first requires differentiation of the speakers recorded or detected during the conversation. This may be done, for example, using the following code:
[W,s,v]=svd((repmat(sum(x.*x,1),size(x,1),1).*x)*x′);
   although many other solutions and methods are possible. According to an embodiment, the algorithm utilizes multiple microphones to differentiate the signals based on spatial location of the speakers. Many devices such as smartphones already have two microphones, including one for background noise reduction, located on opposite sides of the device. Once the sound signals are isolated using this approach, deep neural networks can be used to perform recognition and translation of spoken information into raw text by exploiting a model trained from a large amount of spoken language data. An NLP based classifier with features such as an introduction as a clinician (e.g. “Hello, I am Dr. Smith”), use of complex clinical terms, and beginning with a question (e.g. “How are you feeling today?”) can be used to differentiate which text belongs to a patient, clinician, or care giver. Additionally, a speech act classifier can be used to analyze the conversation structure at various levels (locutionary, illocutionary, and perlocutionary) that would enable effective understanding of the clinical scenario discussed between the patient and clinician/caregiver. The last step is to take in raw text from either source type (document or soundscape) and process the data using the following NLP methods including: (i) contextual analysis including (1) syntactic, (2) semantic, and (3) discourse parsing; (ii) named entity recognition; (iii) temporal resolution; and/or (iv) negation detection, among others.

At optional step 180 of the method, the record collection system or device sends one or more of the individual's existing or prior medical records to the determined location. The record collection system may function in more than one direction, and thus can provide a location or healthcare professional with records about the individual. This may be especially advantageous if the individual is at an office for the first time and must provide a medical history or other background or relevant information, or if the individual is in an emergency situation in which he or she is unable to verbally provide a medical history or other background or relevant information. Accordingly, once the collection system or device has determined that the detected location corresponds to a location where medical records are generated or stored, the system or device sends or otherwise presents a ‘send record’ request to the user. The authorization request may notify the user that it has tentatively or definitely determined that it is located at a place where medical records are generated or stored, and request input from the user as to whether records should be provided to this location.

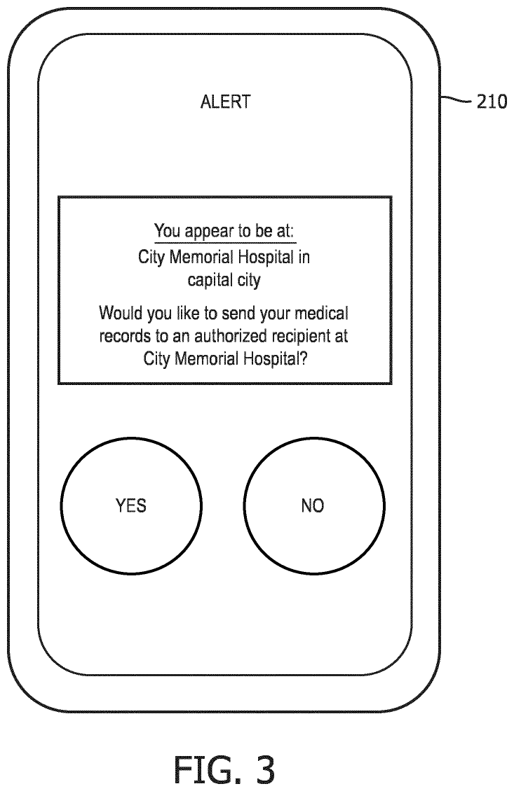

Referring to FIG. 3, in one embodiment, is a schematic representation of a display screen of a record collection device 210. The collection system or device has determined that the detected location corresponds to a location where a healthcare provider may require medical records for the user, and presents the user with an authorization request with an option to send records (“YES”) or not send records (“NO”). The user can respond by pressing YES or NO, or by making some other action. According to another example, the device may be a wearable device such as a smartwatch, smart glasses, augmented or virtual reality device, or other device. The user may observe the alert or notification, and may respond by making a motion, voicing a response, or any other method of providing input to the device or system.

If the user authorizes the device or system to send records to the location, the device or system may send records directly, or may direct an intermediary such as a database, clearinghouse, medical record repository, or other database or location to send the records to the location. According to an embodiment, the record collection system or device may comprise a database of authorized recipients at a plurality of different locations to which to send the records.

At optional step 190 of the method, the record collection system or device analyzes the conversation or received medical record to convert it to a standardized or normalized format, and/or to extract health information from the conversation or record. According to an embodiment, health records such as handwritten notes and scanned documents are converted to raw text. This can be achieved, for example, using optical character recognition methods such as gradient-based learning or CNNs (Convolution Neural Networks). The documents can then be analyzed using natural language processing or other methods to identify and/or extract health information from the conversation or record.

According to an embodiment, the record collection system or device is configured to extract information from unstructured data and a wide variety of other incoming formats. For example, medical information can be extracted from unstructured raw text utilizing NLP to extract context, named entities, important concepts, and/or temporal information. Medical records coming from faxes and handwritten notes may require an additional step of conversion to raw text using optical character recognition. According to an embodiment, a database of processed text can be updated as new medical records are added.

According to an embodiment, the record collection system or device is configured to identify one or more relevant concepts from clinical ontologies and is configured to insert information into templates tailored to forms such as health & physical (H&P) forms. According to an embodiment, clinical ontologies can be extracted from the processed text concepts. Concepts may be matched based on similarity score to a database of categorized concepts from previously acquired H&Ps. There are a number of possible embodiments to match these concepts with the appropriate category. For example, one method is to use RNNs (Recurrent Neural Networks) to learn features of concepts and then use a logistic regression to place those features in the correct category. According to an embodiment, the information can be made useful for the clinician as a summarized but comprehensive history and physical. This can be achieved by using a template based approach with a slot filling task. The data stored in the processed text database or as an H&P may also be used for precision health analytics such as predicting outcomes from future action and interventions or for adverse event risk scoring.

The record collection system or device can be further configured to provide a timeline to the user, as well as end-to-end automated information retrieval workflow. Following a share request, the information can be summarized via a template based approach to generate a patient summary, such as an H&P, for the clinician. The patient will also be able to access paraphrased data displayed in a timeline format. According to an embodiment, the timeline may also require paraphrasing approaches to make the information more understandable to patients.

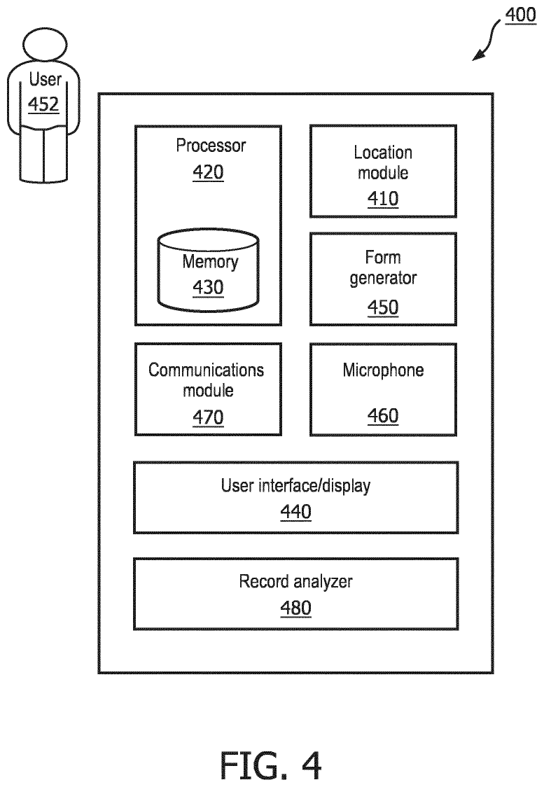

Referring to FIG. 4, in one embodiment, is a schematic representation of a record collection device 400. Record collection device 400 can comprise any of the modules, elements, databases, processors, and/or other components described or otherwise envisioned herein.

According to an embodiment, record collection device 400 comprises a location-aware or location-determination module 410, such as a global positioning system (GPS) receiver. Location module 410 can determine an exact or approximate location and can provide that information to a processor and/or to a user or other remote entity, server, or computer via a communications module. For example, location module 410 can be a GPS receiver that continuously or periodically receives signals utilized to determine a location, and can continuously or periodically provide that information to a user interface and/or to a processor. This allows the system to be location-aware, which provides functionality to the record collection device as described or otherwise envisioned herein.

According to an embodiment, record collection device 400 comprises a processor 420 which performs one or more steps of the method, and may comprise one or more of the modules. Processor 420 may be formed of one or multiple modules, and can comprise, for example, a memory 430. Processor 420 may take any suitable form, including but not limited to a microcontroller, multiple microcontrollers, circuitry, a single processor, or plural processors. Memory 430 can take any suitable form, including a non-volatile memory and/or RAM. The non-volatile memory may include read only memory (ROM), a hard disk drive (HDD), or a solid state drive (SSD). The memory can store, among other things, an operating system. The RAM is used by the processor for the temporary storage of data. According to an embodiment, an operating system may contain code which, when executed by the processor, controls operation of one or more components of the system or device.

According to an embodiment, processor 420 continuously or periodically receives location information from location module 410. The processor automatically determines that the detected location corresponds to a location where medical records are generated or stored. The system comprises or is in communication with a database of locations where medical records are generated or stored, such as hospitals, medical offices, laboratories, and many other types of locations, such as memory 430. Processor 420 may continuously or periodically compare the detected location to the database of locations to identify whenever the user is close to or located at a location where medical records are generated or stored. The system may cross-reference the determined location with the user's schedule, such as knowledge of a medical appointment in a calendar or other schedule, to confirm that the user is at the determined location rather than at a nearby location.

According to an embodiment, record collection device 400 comprises a user interface or display 440 which is configured to provide input to and receive input from user 452. The user interface can be any device or system that allows information to be conveyed and/or received, such as a speaker or screen, among many other types of user interfaces. The information may also be conveyed to and/or received from a computing device or an automated system. The user interface may be located with one or more other components of the system, or may located remote from the system and in communication via a wired and/or wireless communications network.

According to an embodiment, the user interface sends or otherwise presents an authorization request to the user once the collection system or device determines that the detected location corresponds to a location where medical records are generated or stored. The user interface may send or otherwise present a ‘send record’ request to the user once the collection system or device has determined that the detected location corresponds to a location where medical records are generated or stored. An authorization request may notify the user that it has tentatively or definitely determined that it is located at a place where medical records are generated or stored, and request input from the user as to whether records should be requested from and/or provided to the location.

According to an embodiment, record collection device 400 comprises a form generator 450. The form generator may generate a release or authorization form for medical records to accompany a request for those records sent to a location. This can be performed automatically, or in response to input from the user. Automatically generating a release or authorization form, for example, can minimize delays and improve patient/doctor communication. According to an embodiment, the release or authorization need not be a form with labeled text fields, but may be represented by coded transactions using barcodes, RFID or Bluetooth LE transactions (e.g. Apple Pay), QR codes, and other transactions or information. According to another embodiment, the record collection device 400 identifies a form stored in memory. The form may be, for example, pre-authorized for the determined location.

According to an embodiment, record collection device 400 comprises one or more microphones 460 to record a conversation between the individual and the health-care provider at the location. This may be an automated feature when the device determines that it is located at a place where medical records are generated or stored, or may be activated in response to user input. For example, the record collection system or device may prompt the user to record audio as the user enters the location, and/or as the device detects a conversation at the location.

According to an embodiment, record collection device 400 comprises a communications module 470. The communications module facilitates communication between the record collection device and another component of the record collection system, a determined location, a remote server or database, an intermediary, or any other device or location. The communications module may be a wired and/or wireless communications module and may communicate by cellular, Wi-Fi, Bluetooth, IR, radio, or near field communication, among many others.

According to an embodiment, record collection device 400 comprises a record analyzer 480. The record analyzer analyzes a recorded conversation or received medical record to convert it to a standardized or normalized format, and/or to extract health information from the conversation or record. According to an embodiment, health records such as handwritten notes and scanned documents are converted to raw text. This can be achieved, for example, using optical character recognition methods such as gradient-based learning. The documents can then be analyzed using natural language processing or other method to identify and/or extract health information from the conversation or record. Many other methods for analyzing a recorded conversation or received medical record are possible.

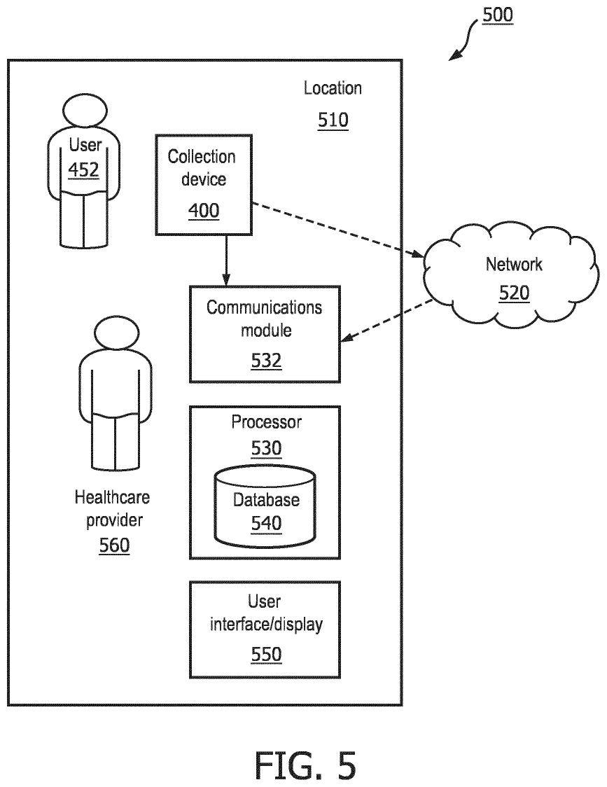

Referring to FIG. 5, in one embodiment, is a schematic representation of a record collection system 500. System 500 can comprise any of the modules, elements, databases, processors, and/or other components described or otherwise envisioned herein.

According to an embodiment, record collection system 500 comprises a user 452 with a record collection device 400. Record collection device 400 may be or comprise any of the record collection device components or elements described or otherwise envisioned herein. For example, record collection device 400 may be a smartphone, computer, wearable device, or any other suitable device. User 452 may be any user of the device, including a patient or a healthcare professional.

According to an embodiment, record collection device 400 comprises a location module that determines the location of the device. The location module may determine, for example, that the device is located near or within a geofence of a location 510 where medical records are likely to be generated or stored. According to an embodiment, record collection device 400 establishes or attempts to establish communication with location 510 to request that medical records be sent to the location and/or be received from the location. The system may comprise a network 520 which is in communication with a communications module 532 of location 510. Communications module 532 may be a component of location 510, or may be a component or element of a device within location 510, such as a server, database, computer, healthcare device, or other component of or within location 510. The network 520 may be any wired and/or wireless network, such as an intranet, the internet, or any other network sufficient for communication as described herein.

According to an embodiment, record collection system 500 comprises a processor 530 which performs one or more steps of the method, and may comprise one or more of the modules. Processor 530 may be formed of one or multiple modules, and can comprise or be in communication with, for example, a database 540 such as a database of medical records. Processor 530 may take any suitable form, including but not limited to a microcontroller, multiple microcontrollers, circuitry, a single processor, or plural processors. Processor 530 may receive a request for medical records from the communications module 532 and the record collection device 400 directly and/or via network 520.

According to an embodiment, record collection system 500 comprises a user interface 550 configured to provide information to and/or receive input from a healthcare professional or provider 560. For example, the user interface may inform the healthcare professional 560 that the user has requested medical records, and may request authorization from the professional to provide those records, a subset of those records, alternative records, or no records. Alternatively, the user interface may provide the healthcare professional 560 with records sent by the user 452 via the record collection device 400.

The user interface can be any device or system that allows information to be conveyed and/or received, such as a speaker or screen, among many other types of user interfaces. The information may also be conveyed to and/or received from a computing device or an automated system. The user interface may be located with one or more other components of the system, or may located remote from the system and in communication via a wired and/or wireless communications network.

### Example 1

According to an example, the record collection method or system described or otherwise envisioned herein may improve a user's treatment. According to this example, a user arrives at a Hospital Emergency Department with labored breathing (dyspena) and chest pain. While in the waiting room, the record collection device determines that the user is in an Emergency department using geolocation. While waiting to be seen, the record collection device gives the user the opportunity to request the data that will be collected today to be automatically shared with his medical record database or account. After being evaluated by a physician, a chest x-ray is ordered. The record collection device then guides the user to radiology. Meanwhile, the user's physician views the user's health information via access granted via the record collection device. The physician notices that the user is high risk for acute decompensated heart failure based on analytics and his data from today's visit. The clinical team begins preparing early with diuretics and vasodilators. They notice he could have a dangerous medication interaction and they change treatment. He receives early treatment thereby preventing extra damage to cardiac tissue. Following his visit to the emergency department, the user sees a cardiologist for long-term care. Before his appointment, he shares his history including his recent emergency visit via the access granted via the record collection device or system. The user handles the data sharing when it is most relevant. He does not need to call in a request later and send in consent forms. The user's medical history is shared seamlessly, removing the step of filling out lengthy forms when he arrives at the Hospital. The user, or anyone else in his care network with the right access privilege can view the user's longitudinal medical data, making it easy for users to keep track of their medical data and take ownership of their own care.

### Example 2

According to an example, the record collection method or system described or otherwise envisioned herein may improve a user's access to and use of medical information. During a visit to an obstetrician, a record collection device records instructions from the physician that the user can refer to later in her journey. Additionally, family history and medical data for electronic medical records are automatically requested, thereby making it easier for the physician to document the encounter. After each sonogram at the imaging center, the images are stored along with corresponding metadata and shared with the obstetrician. At 6 months, an abnormality is detected via ultrasound. The obstetrician is alerted and recommends genetic testing. Again this information is shared with their physician via the record collection system or device. A root cause for the abnormality is discovered and the information is shared with the obstetrician so that they can develop a plan for the complicated delivery. The plan and instructions are saved for both the clinicians and expecting couple. On their delivery day, the obstetrician is out of the country so they must work with another obstetrician who is familiar with their delivery day plan. Using the record collection system or device they are able to easily and accurately transfer information to the new obstetrician. Additionally, the record collection system or device allows them to track the contracts and share with the clinical staff upon arrival. After the baby arrives, the record collection system or device allows them to track the baby's Apgar scores, track excretions, weight, height changes, medications, sleeping, and eating. Moreover, the clinical team has or requests access to their information so that they can ensure they are giving the baby the optimal care. Meanwhile, the record collection system or device allows the mother to track her mood and health.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A record collection device configured to collect medical records for an individual, the device comprising:
a user interface configured to receive input from the individual;
a location module configured to detect a location of the device; and
a processor configured to: (i) automatically determine, by a processor of the record collection device, that the detected location corresponds to a location where a medical record may be generated or stored; (ii) receive, from the individual via the user interface, approval to request the medical record from the determined location; (iii) send, in response to the individual's input, a request to the determined location for the individual's medical record; and (iv) send, in response to input from the individual, one or more prior medical records of the individual to the determined location.

2. The device of claim 1, further comprising a form generator configured to generate a release form or authorization form for the medical records, wherein the sent request comprises the generated form.

3. The device of claim 1, further comprising a microphone, and wherein the processor is further configured to record, using the microphone, a conversation between the individual and a healthcare provider at the location.

4. The device of claim 3, wherein the processor is further configured to send the recorded conversation to a remote server or database.

5. (canceled)

6. The device of claim 1, wherein the processor is further configured to send an authorization request to the individual, wherein the authorization request seeks approval to request the medical record from the determined location.

7. The device of claim 1, wherein the device is in communication with a database comprising information about a plurality of locations where records are generated or stored.

8. A method for obtaining an individual's medical record using a record collection device, comprising the steps of:
automatically detecting, via the record collection device, a location of the individual;
automatically determining, by a processor of the record collection device, that the location corresponds to a location where a medical record for the individual may be generated or stored;
receiving, from the individual, approval to request the generated or stored record from the determined location;
sending, in response to the individual's approval, a request to the determined location for the individual's medical record
sending, in response to input from the individual, one or more prior medical records of the individual to the determined location.

9. The method of claim 8, further comprising the step of receiving, by the record collection device, the requested medical record.

10. The method of claim 9, further comprising the step of sending the received medical record to a remote server or database.

11. (canceled)

12. (canceled)

13. (canceled)

14. The method of claim 8, further comprising the step of extracting information from a received medical record.

15. The method of claim 8, further comprising the step of generating a timeline from the received medical record.

16. The device of claim 3, wherein the processor is further configured to transcribe the recorded conversation to text.

17. The device of claim 3, wherein the processor is further configured to differentiate which text belongs to a patient, clinician, or care giver using an NLP-based classifier with features comprising one or more of an introduction as a clinician, a use of complex clinical terms, and a beginning of a conversation with a question.

18. The device of claim 1, wherein sending the one or more previously requested medical records comprises directing an intermediary to send the one or more records to the determined location.

19. The method of claim 10, further comprising cross-referencing the determined location with a schedule of the individual to confirm that the individual is at the determined location.
